(19) United States
(12) Patent Application Publication
(10) Pub. No.: US 2013/0116733 A1
(43) Pub. Date: May 9, 2013

(54) Bone Clamp and Method

(71) Applicant: Edward Jordan Stoll, JR., Boulder, CO (US)

(72) Inventor: Edward Jordan Stoll, JR., Boulder, CO (US)

(21) Appl. No.: 13/654,026

(22) Filed: Oct. 17, 2012

Related U.S. Application Data
(60) Provisional application No. 61/556,437, filed on Nov. 7, 2011.

(51) Int. Cl. A61B 17/84; A61B 17/88; A61B 17/80

(52) U.S. Cl. 606/282; 606/324

(57) ABSTRACT

A bone clamp and method of using the bone clamp are provided. The bone clamp includes a pair of squeeze handles. Movement of one of the handles forces a push rod in a uniaxial direction. The push rod has a moveable jaw secured to a distal end thereof. A stationary jaw is connected to the other handle member. A curved slot formed in the handle member receives the push rod and ensures a unidirectional or linearly directed force is applied to the push rod. This uniaxial force prevents shifting of the bone plate when the clamp makes contact with a bone and a bone plate. The orientation of the clamping surfaces of the jaws provide an optimal orientation to attach the bone plate to the anterior surface of a bone allowing an unimpeded view of the fracture around the superior and inferior surfaces of the bone.

### CROSS-REFERENCE TO RELATED APPLICATION

The present application claims the benefits of U.S. Provisional Application Ser. No. 61/556,437, filed Nov. 7, 2011, entitled “Bone Clamp and Method” and which is incorporated herein by this reference in its entirety.

### FIELD OF THE INVENTION

The present invention relates to medical devices, and more particularly, to bone clamps and methods of employing bone clamps in medical procedures to repair fractured bones.

### BACKGROUND OF THE INVENTION

Fractured bones often require the use of compression plates to stabilize and set the fractured bone so it may heal. More specifically, a compression plate is used to bridge the fracture, and the plate is secured, such as by screws, into the adjacent bone structure to rigidly support the bone after it has been set by the surgeon. For multiple fractures in a bone, more than one bone plate can be used to stabilize the bone.

In order to install a bone plate, one or more bone clamps are used to precisely hold the bone plate in a desired orientation with respect to the fractured bone. Once the bone plate is secured by screws or other fastening means, the bone clamp(s) is then removed.

Traditional bone clamps resemble forceps in which opposing jaws of a clamp provide the force necessary to hold the bone plate against the bone during the procedure. Traditional bone clamps also include a locking mechanism that locks the jaws against the bone and plate. The locking mechanism may include, for example, a ratchet mechanism or a threaded screw and lug combination that hold the squeeze handles in a stationary position.

As one skilled in the art will appreciate, the surgical area around the fractured bone becomes very crowded with the use of multiple bone clamps along with other surgical instruments being used to hold the tissue open around the bone.

Different types of bone clamps are available with various sized clamping jaws adapted to best clamp the particular bone structure and selected bone plate. However, despite the availability of various shaped and sized clamping jaws, a typical bone repair kit may be inadequate in terms of providing the optimal bone clamp for a particular procedure, thereby limiting the medical practitioner's options in how to orient and otherwise stabilize the fractured bone during attachment of a bone plate.

Another disadvantage of traditional bone clamping devices is that they add problematic weight to the targeted bone structure, making it more difficult to precisely position and secure the bone plate, since the weight of the bone clamp itself can result in a torque or twisting force applied to the plate thereby causing a shifting of the position of the plate with respect to the bone.

Another disadvantage with many bone clamp devices is that bone clamps, because of their size, crowd the medical practitioner's working space and inhibit the practitioner's ability to precisely position the bone plate over the bone structure. The bone clamps may also inhibit the practitioners' view of the fracture when the bone plate is positioned against the bone.

Another significant drawback with prior art bone clamps is that the natural closing action of two jaws rotating about a center axis causes forces to be applied by the jaws of the clamp that are not directed in a unidirectional or linear orientation. Rather, the closing action of the jaws causes forces to be applied that are directed both laterally and axially. Therefore, as a medical practitioner closes the jaw of the clamp around the targeted bone and bone plate, there will inherently be some amount of shifting of the plate against the bone caused by an inherent torque comprising the combination of the forces applied in the different directions.

Therefore, despite the long use of bone clamps in surgical procedures, there is still a need to provide a bone clamp that holds the targeted bone and bone plate such that only a unidirectional or linearly directed force is applied, thereby avoiding the torque or twisting force that occurs with traditional bone clamps. Further, there is still a need to provide a bone clamp that is minimally invasive in terms of its orientation to the targeted bone structure thereby minimizing interference within the surgical area.

### SUMMARY

In accordance with the present invention, a bone clamp and method of installing a bone clamp are provided. The structure of the bone clamp includes a pair of handle members that are squeezed in order to close a pair of opposing jaws around the targeted bone and bone plate. The first and second handle members, also referred to herein as the minor and major handles, are connected to one another and rotate about a central pin.

The second or major handle member receives a straight or linear shaped moveable push rod. The push rod is selectively moved by squeezing or releasing the handles. The minor handle includes an extension in the form of a sleeve which receives push rod and helps to orient the rod for the uniaxial or unidirectional motion.

The minor handle further includes an offset or c-shaped extension that protrudes beyond the sleeve. The distal end of the extension forms a stationary jaw. The distal end of the push rod includes a rotatable or movable jaw that moves toward or away from the stationary jaw by selective squeezing or releasing of the handles.

The end of the major handle that receives the push rod includes a curved slot. The particular geometric orientation of the curved slot with respect to the orientation of the major handle member enables the end of the push rod to remain stationary except for its axial translation, while the major handle member is moved throughout an infinite number of positions. Because the end of the push rod is freely received within the curved slot, squeezing of the handles to advance the push rod results only in an axial force applied to the push rod, thereby avoiding a torque or twisting movement of the jaws which occurs with the traditional bone clamps. The jaws of the bone clamp may be selectively locked by use of a threaded rod and lug combination that is secured to the handles thereby preventing movement of the handles.

According to a method of the invention, a particular construction for the bone clamp is provided including the curved slot which results in the uniaxial application of force to the bone structure and bone plate by the opposing jaws. A user selectively squeezes the handles to precisely position the jaws, thereby providing a consistent compression force against the bone structure and bone plate in order to easily install the bone plate.

Other features and advantages of the invention will become apparent from the following detailed description, taken in conjunction with the claims appended hereto.

### BRIEF DESCRIPTION OF THE DRAWINGS

FIG. 1 is a perspective view of a preferred embodiment of the bone clamp of the present invention;

FIG. 2 is another perspective view of the embodiment of FIG. 1;

FIG. 3 is another perspective view of the embodiment of FIG. 1;

FIG. 4 is a side elevation view of the invention with the jaws illustrated in an open position;

FIG. 5 is another side elevation view, illustrating the push rod actuated thereby moving the movable jaw towards the stationary jaw and to a closed position;

FIG. 6 is an enlarged fragmentary perspective view of the opposing jaws of the bone clamp;

FIG. 7 is another enlarged fragmentary perspective view of the opposing jaws of the bone clamp;

FIG. 8 is an enlarged fragmentary perspective view showing an end of the push rod received in an end of the major handle, and the configuration of the slot and axial opening in end of the handle enabling the handle member to freely move without interference with the end of the push rod so the handle member applies only a unidirectional or linear force to the push rod;

FIG. 9 is a further enlarged fragmentary perspective view similar to FIG. 8 showing the end of the push rod received in the end of the major handle;

FIG. 10 is a fragmentary enlarged perspective view illustrating the major handle member removed in order to view the proximal end of the push rod;

FIG. 11 is a perspective view of a prior art clamp attached to a clavicle bone of a patent;

FIG. 12 is a perspective view of the bone clamp of the present invention attached to the clavicle of a patient.

FIG. 13 is a perspective view of the bone clamp of the present invention attached to the fibula of a patient.

FIG. 14 is a side elevation view of the bone clamp illustrating the jaws in a fully opened position, a coordinate axis system including x and y coordinates, and mathematical expressions that define the geometrical orientation of the curved slot in reference to the position of the handle members;

FIG. 15 is another side elevation view of the bone clamp of FIG. 14, illustrating the jaws in a partially closed position;

FIG. 16 is another side elevation view of the bone clamp of FIG. 14, illustrating the jaws in another partially closed position;

FIG. 17 is another side elevation view of the bone clamp of FIG. 14 illustrating the jaws in a fully closed position;

### DETAILED DESCRIPTION OF THE DRAWINGS

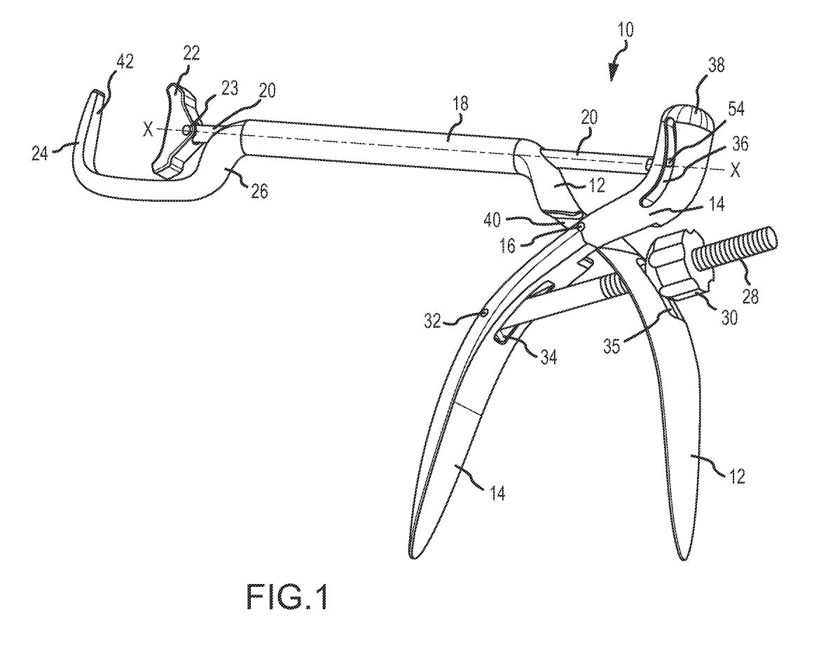

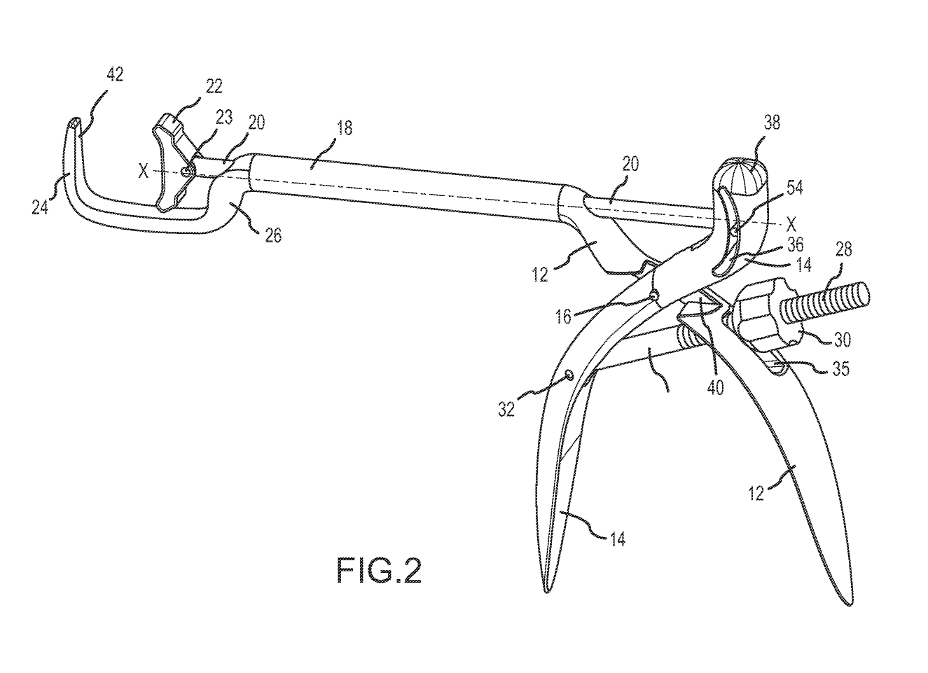

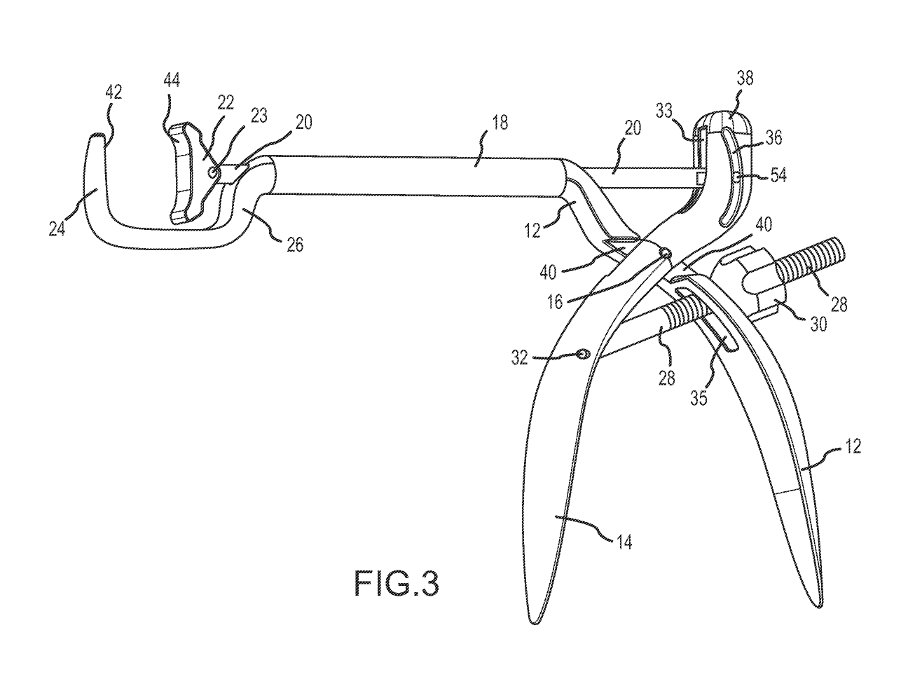

Referring first to FIGS.1-3, the bone clamp device 10 of the invention is illustrated in a preferred embodiment. The clamp 10 includes a first or minor handle member 12, a second or major handle member 14, and the handle members being connected to one another about a central pin 16. The handle members rotate about the central pin 16 when the handle members are squeezed or released.

A distal end of the first handle member 12 includes a tubular shaped sleeve 18 or extension having an internal passageway. A distal end of a sleeve 18 connects to an offset or c-shaped member 26. A distal end of the offset 26 defines a stationary jaw 24. The second or major handle member 14 has a distal end 38 including a transverse curved slot 36 formed through the end 38. A push rod 20 has a proximal end received in an axial opening 33 that communicates with the curved slot 36. As shown in the preferred embodiment, the sleeve or extension has an internal passageway. The distal end of the push rod 20 extends through the passageway of the sleeve 18. The distal end of the push rod 20 has a moveable jaw 22 attached thereto. In the preferred embodiment the jaw 22 is secured as by a pin 23 that enables the moveable jaw to rotate about the pin 23.

FIGS. 1-3 also illustrate a locking mechanism used to lock the position of the jaws with respect to one another. Specifically, the locking mechanism includes a threaded locking bolt 28 having a first end pinned to the handle 14, and a second end that extends through an opening 35 formed in the first handle member 12. A threaded lug 30 is used to lock the position of the handles, thereby also locking the position of the jaws. The first end of the locking bolt 28 is received in a cut-out or slot 34 that enables the threaded bolt 28 to rotate as necessary about pin 32 to thereby accommodate the changing positions of the handle members, and also enables the locking lug 30 to be tightened against the exposed exterior surface of the handle 12. Although the preferred embodiment shows use of a conventional bolt and lug combination for locking the device, it shall be understood that other means can be used to include ratchet systems or other locking systems.

The figures further illustrate a cut-out 40 that is formed in the handle 12 enabling the second handle 14 to rotate between fully opened and closed positions, yet minimizing the profile or size of the handles. Without the cut-out 40, the end 38 of the handle 14 would have to be enlarged to extend towards the minor handle 12 in order to axially align the end 38 with the passageway of the sleeve 18.

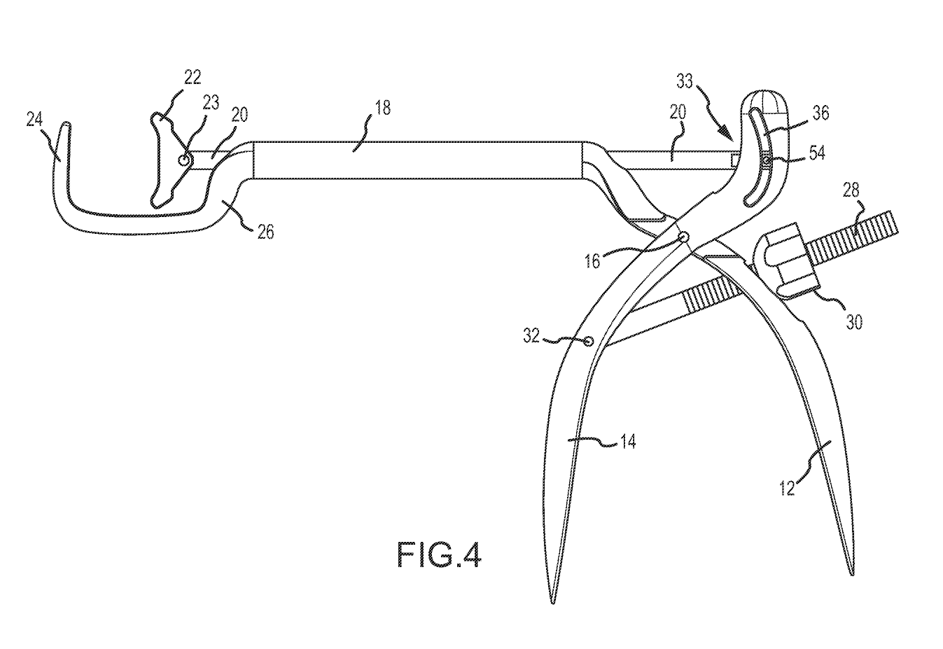

FIG. 4 more specifically illustrates the jaws in a partially open position. In this position, the end of the push rod 20 within the handle 14 is located midway within the axial opening 33 communicating with the slot 36.

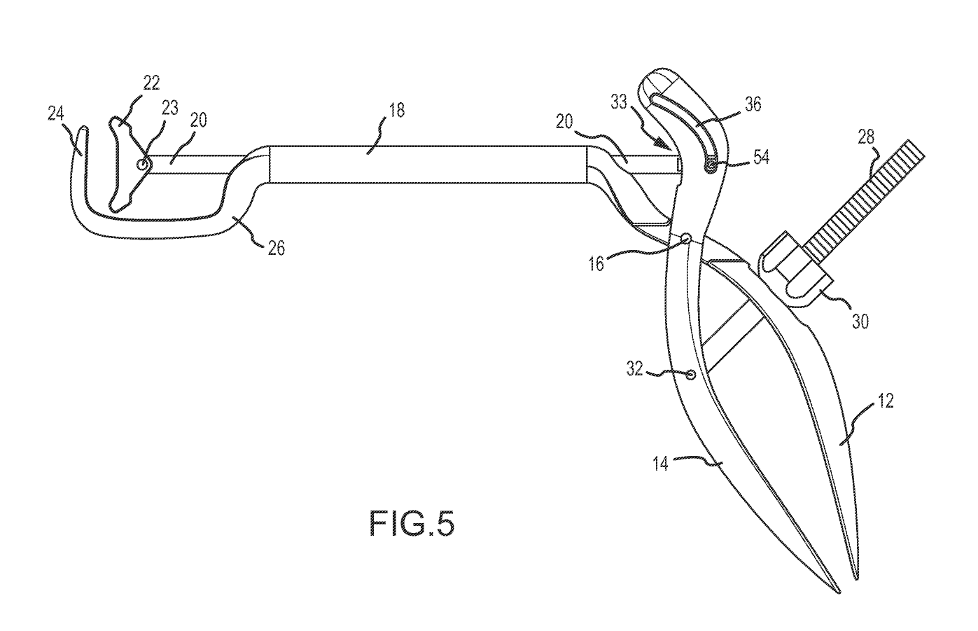

FIG. 5 illustrates the jaws in a closed position, in which the movable jaw 22 is positioned close to the stationary jaw 24, and the handle members being closely spaced from one another. In this position, the end of the push rod 20 within the handle 14 is located at the bottom or lower end of the axial opening 33 communicating with the slot 36.

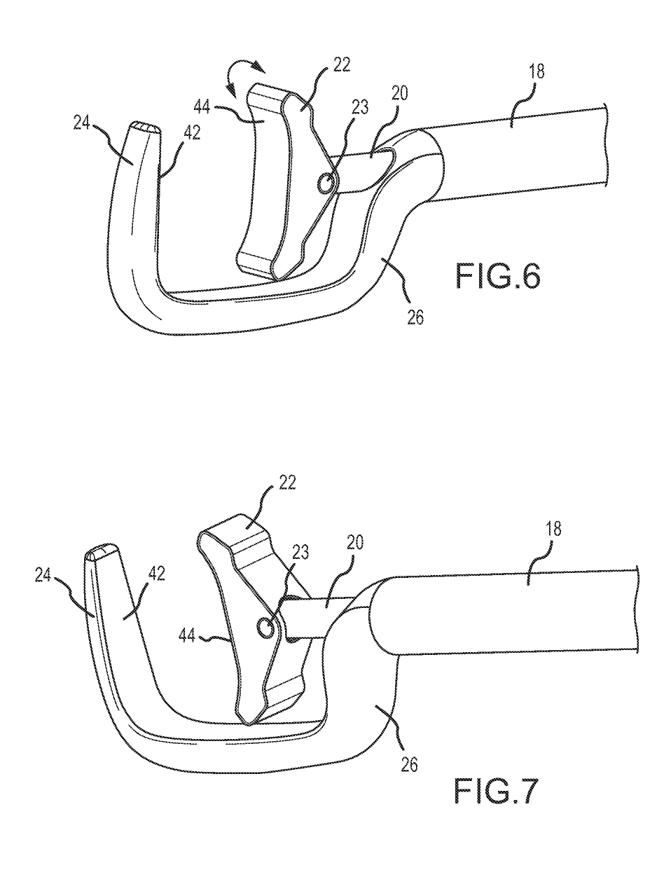

Referring to FIGS. 6 and 7, these enlarged fragmentary perspective views illustrate in better detail a preferred shape for the moveable jaw 22, and its ability to rotate about the pin 23. As compared to the flat engaging surface 42 of the stationary jaw 24, the moveable jaw 22 may have a more curved engaging surface 44, noting particularly the curvature found adjacent the lower and upper portions of the engaging surface 44.

In use, it may be advantageous to first position the posterior side of the bone against the stationary jaw 42, and then squeeze the handle so that the moveable jaw makes contact with the anterior side of the bone, which is typically the side of the bone that receives the bone plate for clavicle fractures. The rotational ability of the moveable jaw helps to ensure that when contact is made with the moveable jaw against the anterior side of the bone plate, the moveable jaw naturally centers over the surface being contacted.

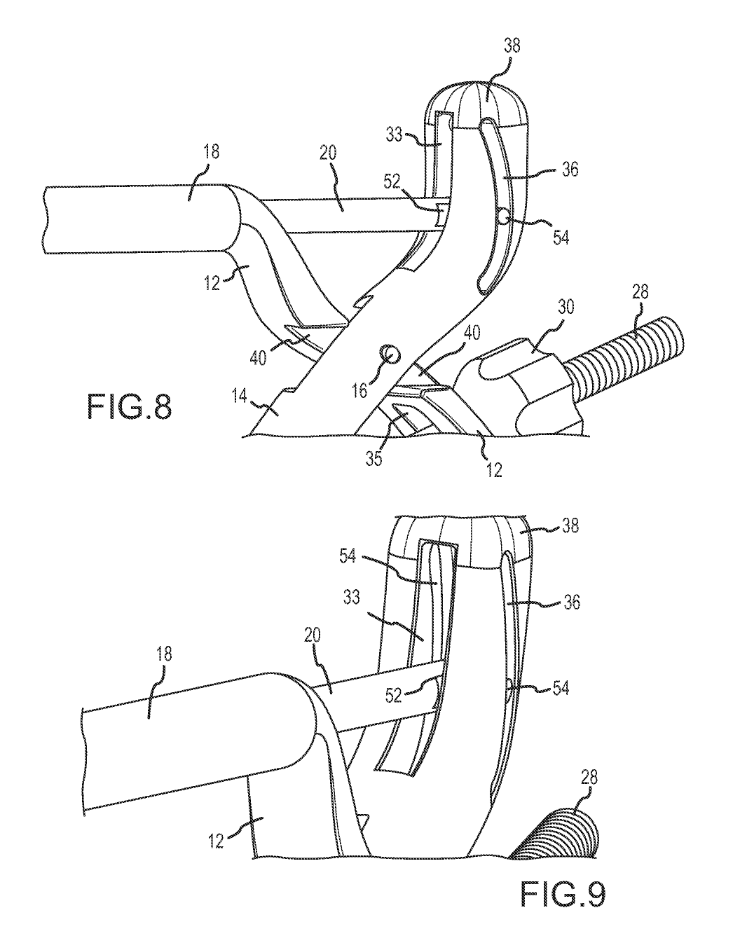

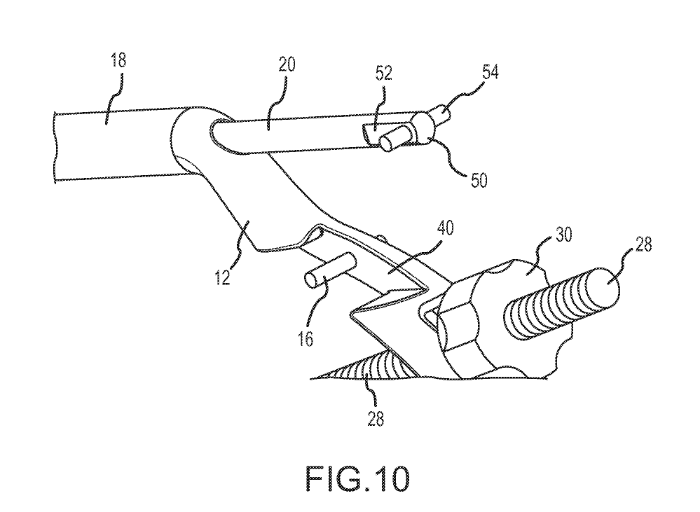

FIGS. 8-10 show the orientation of the axial opening 33 and the slot 36 which enables the major handle member 14 to be moved to infinite positions, yet minimizing any torque or twisting forces upon the axially directed push rod 20. Referring to FIG. 10, the end 50 of the push rod is sized and shaped to clear the axial opening 33, and the only part of the push rod that makes contact within the handle is the transverse pin 54 against the edges of the slot 36. FIG. 10 also shows a chamfer or cut plane 52 formed on one or both sides of the push rod 20 enabling the end 50 to be inserted within the axial opening 33 without contact, and the transverse pin 54 is positioned through an opening in the proximal end 50. This configuration of the proximal end 50 of the push rod 20 minimizes frictional contact between the handle member 14 and push rod 20, enabling the handle member 14 to be squeezed with minimal force and allowing the handle member to move without imparting any appreciable torque or twisting force upon the rod 20. To improve slidable contact between the pin 54 and the edges of the groove 36, the groove or pin may be treated with a sterile lubricant, or may be coated with a material such as Teflon®.

One important advantage of the present invention is the ability to provide a bone clamp with traditional squeeze handles but generating clamping force with the jaws in which only a unidirectional force is applied by the jaws against the targeted bone and hardware. As mentioned, this unidirectional movement of the moveable jaw prevents the clamping device from inadvertently twisting the bone or bone plate, which otherwise may cause misalignment between the bone plate and bone, or worse, could further damage the fractured bone.

Another important advantage of the present invention is the orientation of the jaws 22 and 24 with respect to a targeted bone structure such as a clavicle bone. As mentioned, it becomes more difficult for a medical practitioner to successfully stabilize and secure a bone plate to a bone when the bone clamp interferes with the medical practitioner's working space and view of the fractured bone.

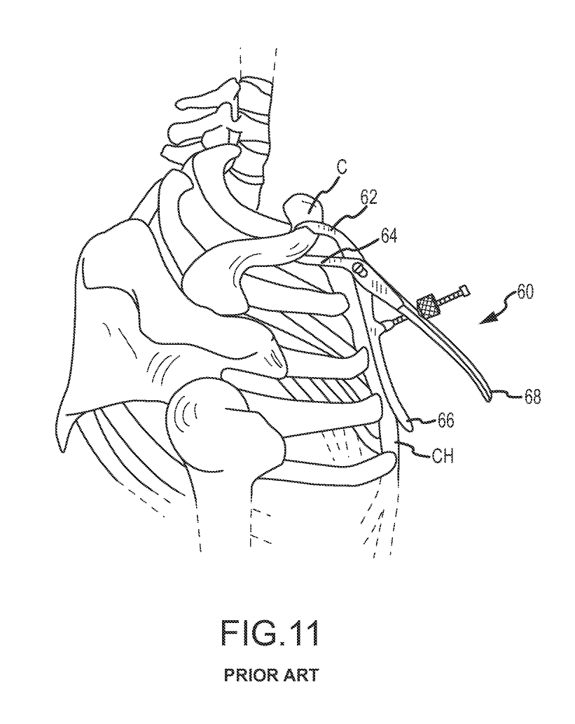

Referring now to FIG. 11, a prior art bone clamp 60 is illustrated as it may be applied to the clavicle bone C. Based upon the orientation of the jaws 62 and 64, the bone clamp 60 has both of the handles 66 and 68 that extend close to, or perhaps in contact with, the patient's chest CH. Depending upon the size of the patient, often times the chest of the patient makes it very difficult for the jaws 62 and 64 to clamp the superior and inferior surfaces of the clavicle bone, noting that proper clamping with use of the device 60 requires superior and inferior contact due to the angular orientation of the jaws 62 and 64 and the shape of the contact surfaces of the jaws.

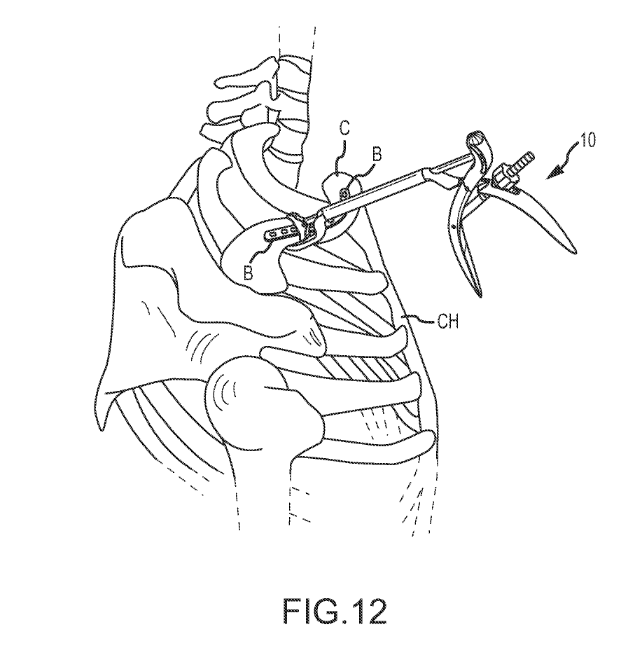

Referring now to FIG. 12, the bone clamp 10 of the present invention is illustrated with respect to clamping the same clavicle bone C, and wherein a bone plate B is secured to the anterior side of the clavicle C. For a great majority of clavicle fractures, an anterior oriented bone plate is used. Therefore, it is highly advantageous to make use of a bone clamp in which the jaws of the clamp are positioned to face the posterior and anterior sides of the bone, as opposed to the superior and inferior surfaces of the bone. With the device 10, a precisely controlled unidirectional compression force may be applied against the bone and bone plate to more easily secure the bone plate to the bone without shifting of the plate. As can be appreciated by review of the prior art clamp of FIG. 11, there is very little surface area, if any, of the jaws 62 and 64 that are capable of holding an anterior positioned bone plate. Therefore, there is a much greater chance of the bone plate shifting when using a traditional bone clamp.

In comparing the orientation of the prior art clamp of FIG. 11 with the clamp 10 of the present invention shown in FIG. 12, it is also apparent that the orientation of the clamp 10 is shifted approximately by 90 degrees as compared to the prior art clamp 60, that therefore opens up available working space directly around the surgical area for the practitioner, as well as providing the optimal anterior/posterior contact configuration for securing the bone plate B to the clavicle C. Further, the posterior/anterior orientation of the jaws allow the practitioner to view the bone fracture on either the superior or inferior surfaces of the clavicle to ensue the plate has adequately stabilized the optimum set position for the bone. In the prior art clamp of FIG. 11, when an anterior bone plate is used along with the jaws of the clamp oriented to contact the superior and inferior clavicle surfaces, this greatly inhibits the practitioner's ability to view the bone fracture and to determine if the bone is set and if the bone plate is in the optimal position to stabilize the fracture.

Referring now to FIG. 13, the bone clamp 10 of the present invention is illustrated with respect to clamping the fibula bone F, which is adjacent the tibia bone T on the leg of a patient. In this case, a bone plate B is also secured to the anterior side of the fibula bone F. Like use of the device with clamping a clavicle, there are advantages to use of the bone clamp in which the jaws of the clamp are positioned to face the posterior and anterior sides of the bone. Accordingly, with the device 10, it is capable of precisely controlling the unidirectional compression force against the bone F and the bone plate B to more easily secure the bone plate against the bone without shifting of the plate. Also, the FIG. 13 shows that the orientation of the clamp is also shifted approximately 90 degrees as compared to the prior art clamp 68 which was used to clamp the clavicle. Accordingly, the working space directly around the surgical area is more open, as well as providing the optimal anterior/posterior contact configuration for securing the bone plate B to the fibula F. Also, the posterior/anterior orientation of the jaws of the device allows the practitioner to view the bone fracture on either the superior or inferior surfaces of the fibula to ensure the plate has adequately stabilized the optimum set position for the bone.

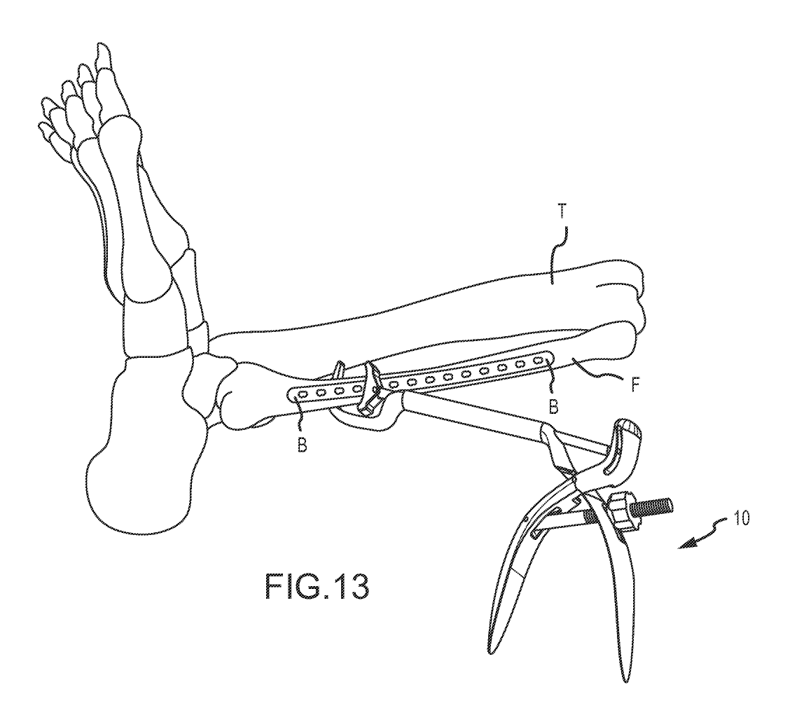

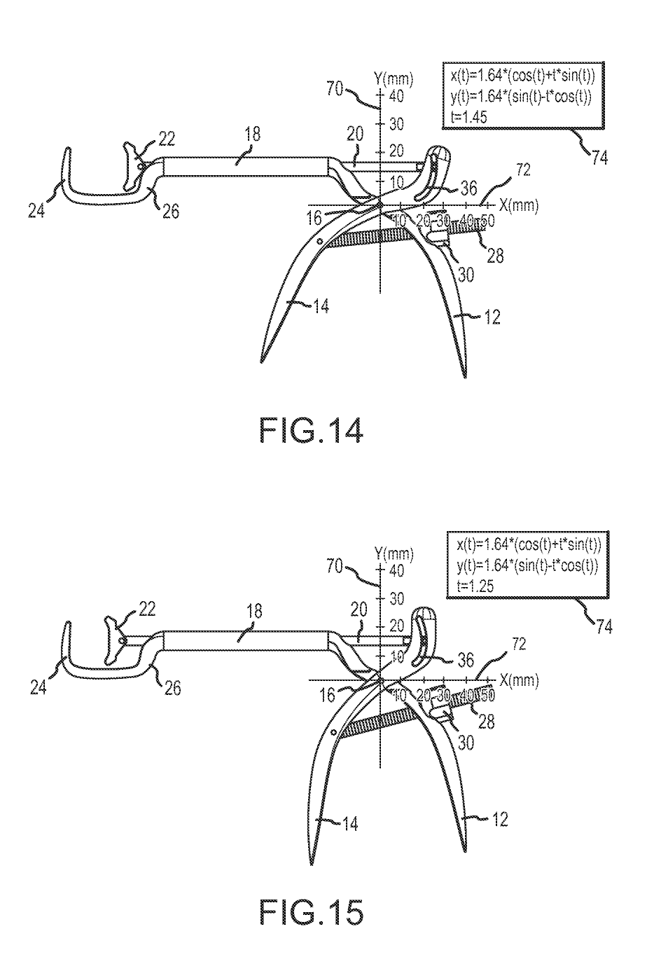

Now referring to FIG. 14, the device 10 is illustrated with a coordinate axis system having an origin of the coordinate axis system located at the central pin 16, and the x and y axes extending as shown. The coordinate axis system includes a y axis 70, an x-axis 72, and an origin defined at the central pin 16. General dimensions are also provided on the coordinate axis system indicating a general preferred size for the clamp for applications such as clamping a clavicle; however, it shall be understood that these dimensions are provided for purposes of disclosing the preferred embodiment generally, it being understood that the clamp 10 could be made larger or smaller depending upon its intended use.

FIG. 14 also illustrates mathematical expressions 74 that can be used to describe the optimal geometric orientation of the axial opening 33 and slot 36 that allows for travel of the handle member 14 to prevent torque from being applied against the push rod 20, and thereby ensuring that the force applied to the push rod 20 is only in the unidirectional orientation along the x-x axis. One goal for the slot 36 is to provide it in such a way so that there is minimal “slop” with the transverse pin 54. That is, defining a precise location for the slot 36 enables the slot 36 to be very small for receiving a very small pin, and thereby minimizing torque applied to the push rod 20 that might otherwise occur if the slot 36 was not precisely defined in favor of a generally large slot.

Therefore, mathematically determining the optimal location of the slot 36 with respect to the location of the handles 12 and 14 provides a great advantage. Generally, the mathematical expressions 74 can be derived by determining the length of an arc that extends from a tangent point on a circle. Multiple arc lengths are determined with multiple corresponding tangent points defined by angles measured from the center of the circle. A circle involute is then created by locating points at the end of each arc length. This circle involute then corresponds to the optimal coordinate positions of the slot 36 as discussed below.

The device 10 is first shown in the FIG. 14 with the handle members fully spaced from one another; thereby resulting in the jaws placed in a fully open position or retraced position. T is the variable in the mathematical expressions, and when assigning a value of 1.45 to T, solving the mathematical expressions provides the position coordinates x and y for the slot location which must be made available to receive the transverse pin 54. Thus for FIG. 14, the position coordinates x and y are located where the pin 54 is shown on the coordinate system.

Now referring to FIG. 15 in another example, the handle members are partially moved towards one another, resulting in the jaws moving to a more closed position. This position of the jaws can also be mathematically correlated in accordance with the mathematical expressions 74. In this example, the position of the transverse pin 54 within the curved slot 36 is now determined by the variable T having a value of 1.25. As compared to FIG. 14, the movement of the handle member 14 results in the pin 54 being located in the optimal position within the slot 36 at a point lower than shown in the FIG. 14.

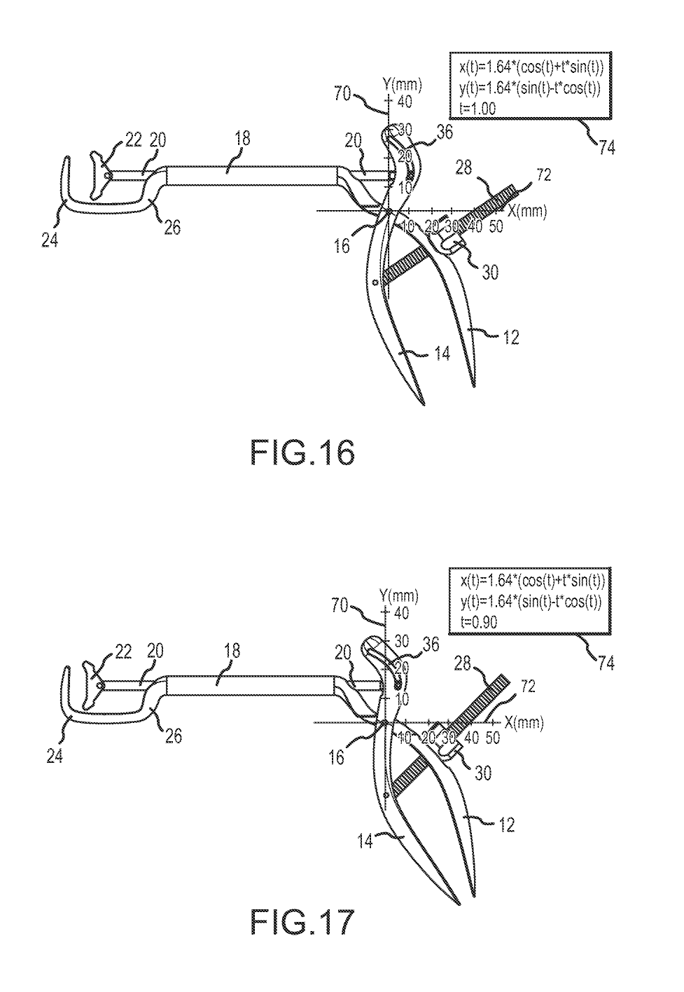

Referring to FIG. 16 in another example, the handle members are moved yet closer to one another resulting in the push rod being further translated so that the moveable jaw 22 is placed in a closer position to the stationary jaw 24. The particular coordinate position of the transverse pin 54 in the curved slot 36 for this example is again determined by use of the expressions 74. In this position, the optimal coordinate location of the slot 36 can be defined when the value of T is 1.0.

Finally, referring to FIG. 17, the handle members are now shown squeezed close together, resulting in the moveable jaw 22 being placed yet closer to the stationary jaw 24. In this position, the optimal coordinate location of the slot 36 can be defined when the value of T is 0.90.

With these four example positions of the device, it can be seen that optimal coordinate locations for the slot 36 can be defined in terms of a continuous curve or arc formed by connecting a number of coordinate points generated according to the mathematical expressions 74. Thus, the transverse pin 54 may be received in the curved slot to minimize any potential torque applied to the push rod 20 along an infinite number of handle positions.

Although the preferred embodiment illustrates a continuous tubular shaped sleeve 18, the sleeve 18 could take other forms. For example, in lieu of a sleeve, a linear extending member could be provided with a plurality of separated tubular sections mounted thereto to receive the rod 20, and these sections would still effectively guide the push rod 20. Alternatively, an extension of the handle terminating in the stationary jaw could have some other type of retaining element(s) such as a plurality of spaced rings, or a groove or channel along the length of the extension that receives a flange or a plurality of extensions from the rod. These alternative configurations would each still be capable of controlling displacement of the rod along an axis to achieve the linear displacement of the rod so that force applied by the movable jaw would be unidirectional without torque or twisting applied to the bone or a bone plate positioned over the bone.

Although a preferred embodiment is illustrated in use with respect to repair of the clavicle, it shall be understood that the bone clamping device of the present invention has utility with respect to many other bone clamping requirements, for example, and not limited to, clamping of a bone plate against the fibula for repair of the fibula.

What is claimed is:

1. A bone clamp comprising:
a first handle member;
a second handle member connected to the first handle member;
said first handle member having a sleeve with an internal passageway, and a distal end of said sleeve having a first jaw;
said second handle member having a distal end including a transverse curved slot; and
a push rod having a proximal end communicating with said transverse curved slot, a distal end of the push rod extending through the passageway of the sleeve, and the distal end having a second jaw attached thereto.

2. A bone clamp comprising:
a first handle member;
a second handle member connected to the first handle member about a central pin;
said first handle member having a sleeve with an internal passageway, and a distal end of said sleeve having a stationary jaw;
said second handle member having a distal end including a transverse curved slot, and an axial opening communicating with said curved slot; and
a push rod having a proximal end received in the axial opening, a distal end of the push rod extending through the passageway of the sleeve, and the distal end having a moveable jaw attached thereto.

3. A bone clamp, as claimed in claim 2, wherein:
said proximal end of said push rod has a transverse pin extending into said transverse curved slot.

4. A bone clamp, as claimed in claim 2, further including:
a locking mechanism secured to the first and second handle members for locking a position of the jaws with respect to one another, said locking mechanism including a threaded locking bolt secured to one of said first and second handle members and a threaded lug attached to the threaded locking bolt, and said threaded lug being selectively positionable for locking the position of the handles.

5. A bone clamp, as claimed in claim 2, wherein:
said transverse curved slot has a shape such that squeezing of the handle members results in said proximal end of said push rod receiving a unidirectional force from said second handle member; and
wherein second handle member freely moves about said proximal end of said push rod resulting in different portions of said transverse curved slot being contacted with said proximal end of said push rod.

6. A bone clamp, as claimed in claim 5, wherein:
said transverse curved slot has a shape according to a mathematical expression defined in terms of a coordinate axis system including x and y coordinates, and said mathematical expression being defined as x(0=1.64*(cos(t)+t*sin(t)) and y(t)=1.64*(sin(t)−t*cos(t)).

7. A method of stabilizing a fractured bone, said method comprising:
(a) providing a bone clamp comprising:
(i) a first handle member; (ii) a second handle member connected to the first handle member about a central pin; (iii) said first handle member having a sleeve with an internal passageway, and a distal end of said sleeve having a stationary jaw; (iv) said second handle member having a distal end including a transverse curved slot, and an axial opening communicating with said curved slot; and (v) a push rod having a proximal end received in the axial opening, a distal end of the push rod extending through the passageway of the sleeve, and the distal end having a moveable jaw attached thereto; and
(b) squeezing the handle members to close the jaws around a bone, wherein said transverse curved slot moves with respect to said proximal end of said push rod resulting in a unidirectional force being exerted against the push rod for linear movement of said push rod through said passageway.

8. A method as claimed in claim 7, wherein:
said jaws of said bone clamp are oriented to contact the anterior and posterior sides of the bone.

9. A method as claimed in claim 7, wherein:
a compression plate is placed adjacent the bone, and said jaws are squeezed to hold the compression plate against the bone.

10. A method, as claimed in claim 7, wherein:
said bone is a clavicle bone.

11. A method, as claimed in claim 7, wherein:
said bone is a fibula bone.

12. A method, as claimed in claim 7, further including:
locking the jaws in a closed position against the bone by use of a locking mechanism incorporated on said bone clamp.

13. A method, as claimed in claim 7, wherein:
said moveable jaw is rotatable about a pin for selective adjustment of said moveable jaw as it contacts the bone during the squeezing of the handle.

14. A method of stabilizing a fractured bone, said method comprising:
(a) providing a bone clamp comprising:
(i) a first handle member; (ii) a second handle member connected to the first handle member (iii) said first handle member having a distal end including a stationary jaw; (iv) said second handle member having a distal end; and (v) a push rod having a proximal end communicating with the distal end of the second handle member, a distal end of the push rod extending toward the stationary jaw, and the distal end of the push rod having a moveable jaw attached thereto; and
(b) squeezing the handle members to close the jaws around a bone, wherein said push rod moves along an axis in linear movement such that the moveable jaw applies a unidirectional force without torque applied to the bone.

15. A method as claimed in claim 14, wherein:
said jaws of said bone clamp are oriented to contact the anterior and posterior sides of the bone.

16. A method as claimed in claim 14, wherein:
a compression plate is placed adjacent the bone, and said jaws are squeezed to hold the compression plate against the bone.

17. A method, as claimed in claim 14, wherein:
said bone is a clavicle bone.

18. A method, as claimed in claim 14, wherein:
said bone is a fibula bone.

19. A method, as claimed in claim 14, further including:
locking the jaws in a closed position against the bone by use of a locking mechanism incorporated on said bone clamp.

20. A method, as claimed in claim 14, wherein:
said moveable jaw is rotatable about a pin for selective adjustment of said moveable jaw as it contacts the bone during the squeezing of the handle.

21. A bone clamp comprising:
a push rod;
a first handle member communicating with the push rod;
a second handle member connected to the first handle member;
said first handle member having an extension with means for controlling displacement of the push rod, and a distal end of said extension having a first jaw;
said second handle member having a distal end including a transverse curved slot; and
said push rod has a proximal end communicating with said transverse curved slot, a distal end of the push rod extending along the extension of the first handle member, and a distal end of the push rod having a second jaw attached thereto; and
wherein said push rod moves along an axis in linear movement when the first and second handle numbers are squeezed towards one another such that the second jaw applies a unidirectional force against a targeted object without torque.
